(19) United States
(12) Patent Application Publication
(10) Pub. No.: US 2022/0238236 A1
(43) Pub. Date: Jul. 28, 2022

(54) ESTIMATING NUMBERS OF PATIENTS TREATED FOR EACH OF MULTIPLE MEDICAL CONDITIONS BASED ON AMOUNTS OF MEDICINES ADMINISTERED

(71) Applicant: IMS Software Services, Ltd., Wilmington, DE (US)

(72) Inventors: Xiaojun MA, Tokyo (JP); Shuichi BEPPU, Tokyo (JP); Matsuru YAMAZAKI, Tokyo (JP); Osamu FUJITA, Chigasaki-shi (JP); Genryou UMITSUKI, Kashiwa-shi (JP)

(21) Appl. No.: 17/216,025

(22) Filed: Mar. 29, 2021

(30) Foreign Application Priority Data
Jan. 28, 2021 (JP) 2021-012266

(51) Int. Cl. G16H 50/70; G16H 50/20; G16H 70/40; G16H 10/60; G16H 20/10; G16H 40/20; G16H 70/20; G06N 20/00

(52) U.S. Cl. CPC G16H 50/70; G16H 50/20; G16H 70/40; G06N 20/00; G16H 20/10; G16H 40/20; G16H 70/20; G16H 10/60

(57) ABSTRACT

Methods and systems to train a global model to estimate numbers of patients treated for each of multiple medical conditions by a medical facility, based on medicines administered by the medical facility. Training of the model may be tailored for a situation in which a first one of the medicines is administered for a plurality of the medical conditions and a second one of the medicines is administered for a subset of the plurality of medical conditions. Where the medicines include a general medicine administered for a plurality of the medical conditions, and one or more exclusive medicines, each administered for a respective one of the plurality of medical conditions, parameters of the model may be modified for the selected medical facility based a ratio at which the selected medical facility administers the general medicine amongst patients of a plurality of the diseases.

### CROSS-REFERENCE TO RELATED APPLICATION

This application claims benefit and priority to Japanese Patent Application No. 2021-012266, filed Jan. 28, 2021, entitled, “INFORMATION PROCESSING APPARATUS, INFORMATION PROCESSING METHOD, AND PROGRAM” incorporated by reference in its entirety.

### BACKGROUND

When a pharmaceutical company conducts sales activities with a medical facility, it is useful to have a grasp on the number of patients per disease in each medical facility.

### SUMMARY

Disclosed herein are methods and systems to train a global model to estimate numbers of patients treated for each of multiple medical conditions by a medical facility, based on amounts of medicines administered to the patients by the medical facility.

Training of the model may be tailored for a situation in which a first one of the medicines is administered for a plurality of the medical conditions and a second one of the medicines is administered for a subset of the plurality of medical conditions.

Where the medicines include a general medicine administered for a plurality of the medical conditions, and one or more exclusive medicines, each administered for a respective one of the plurality of medical conditions, parameters of the model may be modified for the selected medical facility based a ratio at which the selected medical facility administers the general medicine amongst patients of a plurality of the diseases.

### BRIEF DESCRIPTION OF THE DRAWINGS

FIG. 1 is a block diagram of an information processing device.

FIG. 2 is a block diagram of the information processing device in a learning phase.

FIG. 3 is a block diagram of the information processing device in an estimating phase.

FIG. 4A is a table of medical facility data.

FIG. 4B is a table of indication data.

FIG. 4C is a table of drug usage amounts.

FIG. 5 is an illustration of a model.

FIG. 6 is a table of parameters of the model of FIG. 5.

FIG. 7 is a flowchart of a method of training a model (i.e., a learning phase).

FIG. 8 is a flowchart of a method of using a model (i.e., an estimating phase).

FIG. 9 is an illustration of the model of FIG. 5 according to a first variation.

FIG. 10 is an illustration of an adjustment model used in a second variation.

FIG. 11 is an illustration of an estimating phase of the second variation.

### DESCRIPTION

An embodiment is described in detail hereinbelow with reference to attached drawings. Note, the following embodiments do not limit the invention according to the Scope of patent Claims, and not all the combinations of features described in the embodiments are essential to the invention. Two or more features of the plurality of features described in the embodiments may be combined arbitrarily. Further, identical or similar configurations are given the same reference numbers, and duplicate descriptions are omitted.

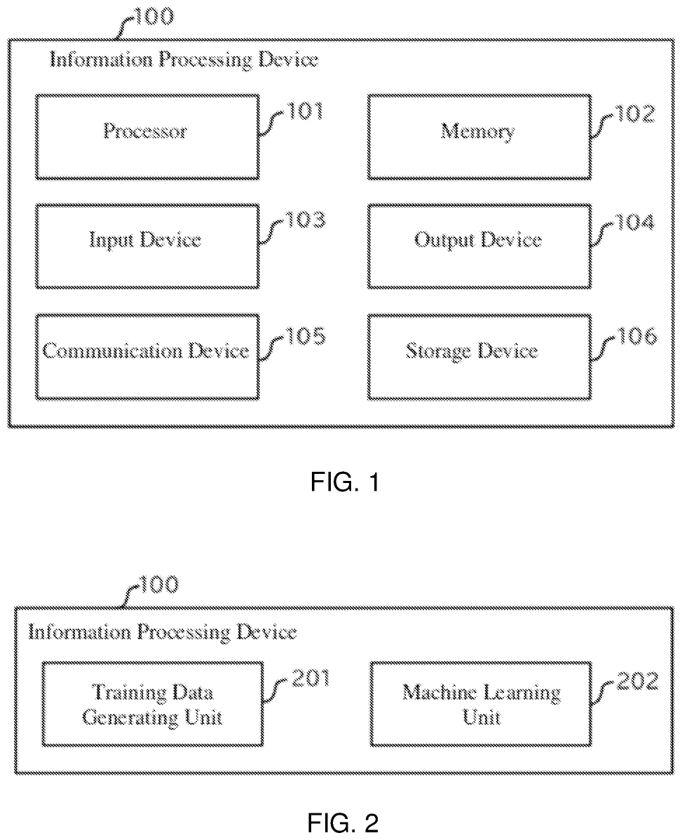

A hardware configuration of an information processing device 100 according to the one embodiment of the present invention is described with reference to the block diagram in FIG. 1. The information processing device 100 can execute both machine learning (called learning phase hereinbelow) to create a model for estimating a number of patients of each of a plurality of diseases included in a defined disease group per medical facility, and estimation (called estimating phase hereinbelow) of a number of patients for which the model is used. In the following embodiment, patient of a defined disease refers to a person affected by a defined disease who is actually receiving treatment at a medical facility (for example, taking a drug). Medical facility refers to a facility that provides people with a medical practice, including, for example, a hospital, clinic, medical office, and the like. Disease group refers to a group constituted of a plurality of related diseases.

The information processing device 100 is realized by an information processing device such as, for example, a PC or a workstation, smart phone, or tablet device. The information processing device 100 may be realized by a single device, or may be realized by a plurality of devices interconnected via a network. The learning phase and the estimating phase may be carried out by the same information processing device 100 or may be carried out by a separate information processing device 100.

The information processing device 100 has each of the constituent elements illustrated in FIG. 1. A processor 101 controls operation of the entire information processing device 100. The processor 101 is realized by, for example, a CPU (central processing unit), or a combination of a CPU and a GPU (graphics processing unit), or the like. A memory 102 stores a program used in operation of the information processing device 100, temporary data, or the like. The memory 102 is realized by, for example, a ROM (read only memory), a RAM (random access memory), or the like.

An input device 103 is used by a user of the information processing device 100 to perform input to the information processing device 100, and is realized by, for example, a mouse, a keyboard, or the like. An output device 104 is used by the user of the information processing device 100 to confirm output from the information processing device 100, and is realized by, for example, an output device such as a display, or an audio device such as a speaker. A communication device 105 provides a function whereby the information processing device 100 communicates with another device, and is realized by, for example, a network card or the like. Communication with the other device may be wired communication or may be wireless communication. A storage device 106 is used to store data used in processing of the information processing device 100, and is realized by, for example, a HDD (hard disk drive), a SSD (solid state drive), or the like.

A functional configuration for the information processing device 100 to execute the learning phase is described with reference to the block diagram in FIG. 2. When executing the learning phase, the information processing device 100 may have the functional blocks illustrated in FIG. 2.

A training data generating unit 201 may generate training data used in machine learning. A machine learning unit 202 generates a model for estimating a number of patients of each of a plurality of diseases included in a defined disease group by performing machine learning using training data generated by the training data generating unit 201. Operation of the functional blocks in FIG. 2 is described in detail hereinbelow.

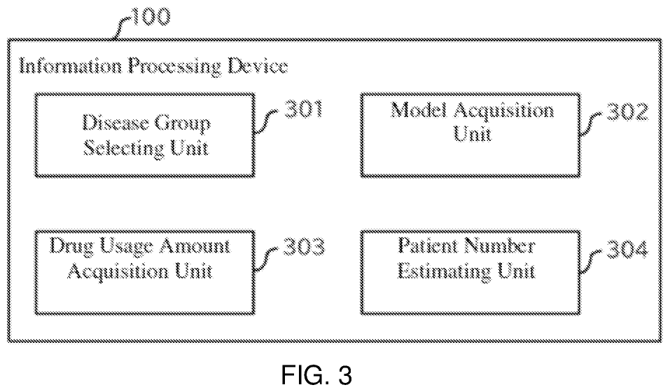

A functional configuration for the information processing device 100 to execute the estimating phase is described with reference to the block diagram in FIG. 3. When executing the estimating phase, the information processing device 100 may have the functional blocks illustrated in FIG. 3.

A disease group selecting unit 301 selects a target disease group to estimate a number of patients. A model acquisition unit 302 acquires a model unique to the disease group selected by the disease group selecting unit 301. This model may by generated in the learning phase. A drug usage amount acquisition unit 303 acquires a usage amount of a drug in a target medical facility to estimate a number of patients. Use of a drug may be any aspect including administration of a drug in a medical facility, prescription of a drug in a medical facility, and sale of a drug in an outpatient facility (for example, a pharmacy) following issuance of a prescription by a medical facility. A sales amount of a drug for an outpatient facility located near a medical facility may be considered to be a usage amount of a drug in the medical facility. A patient number estimating unit 304 estimates a number of patients in the medical facility for each of a plurality of diseases included in a disease group by applying a usage amount of a drug in an individual medical facility to an acquired model. Operation of the functional block in FIG. 3 is described in detail hereinbelow.

Each functional block in FIG. 2 and FIG. 3 may be realized by, for example, the processor 101 executing a command included in a program stored in the memory 102. In place thereof, at least a portion of the functional blocks in FIG. 2 and FIG. 3 may be realized by a dedicated integrated circuit such as an ASIC (application defined integrated circuit) or a FPGA (field programmable gate array).

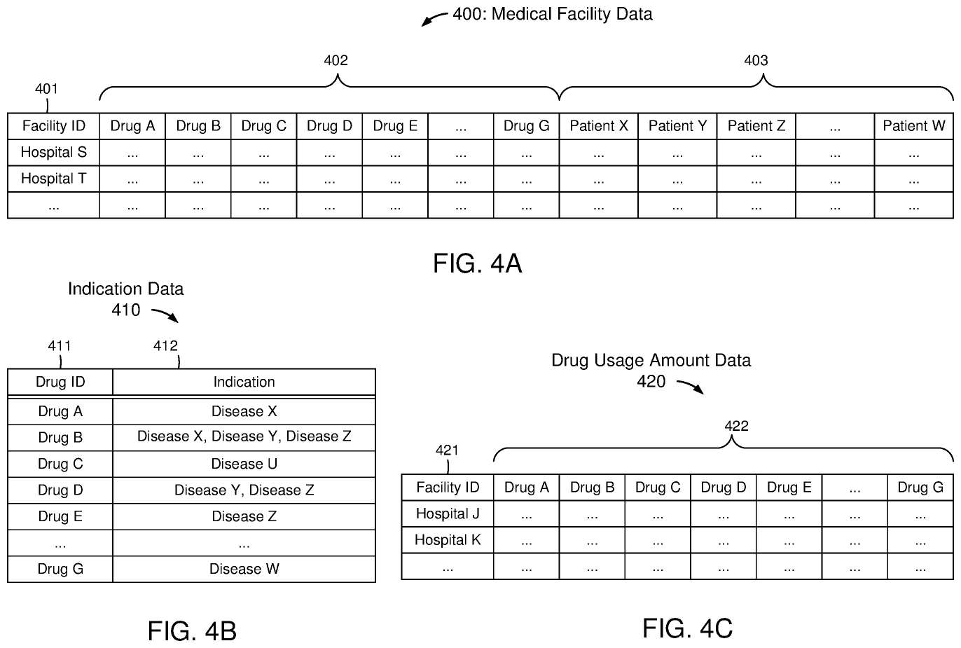

Data used in the learning phase and the estimating phase are described with reference to FIG. 4. These data may be stored in the storage device 106, and these data may be read from the storage device 106 when each functional block of the information processing device 100 is used. In place thereof, these data may be stored in an external storage device, and these data may be received from an external storage device when each functional block of the information processing device 100 is used.

Medical facility data 400 expresses a usage amount of each of a plurality of drugs in individual facilities and a number of patients of each of a plurality of diseases in the medical facility. The medical facility data 400 may be generated by, for example, an interview survey of a medical facility, or analysis of health reports. The medical facility data 400 has an entry per medical facility.

A column 401 expresses an identifier for uniquely identifying a medical facility. A column 402 expresses a usage amount of each of a plurality of drugs in an individual medical facility. The usage amount may be expressed as an arbitrary amount having a significant correlation to the amount used, such as an amount of an active ingredient, an amount by weight, a number of a tablet, and a drug price. A column 403 expresses a number of patients of each of a plurality of diseases in each medical facility. A same type of a drug may be used by an individual patient a plurality of times, so the number of patients is typically a cumulative total number of people. In place thereof, a number of patients may be expressed by an actual number of people. A usage amount of a drug and a number of patients may be a value in a defined duration of time (for example, one month).

In the medical facility data 400, drugs may be classified by any criteria. For example, drugs may be classified by active ingredient. In this case, when, for example, the active ingredient is “metformin”, drugs are classified as the same drug regardless of strength (for example, 500 mg or 250 mg), and they are classified as the same drug regardless of whether they are an original drug or a generic drug. A drug is classified as a separate drug when the active ingredient thereof is not “metformin” (for example, “etanercept”). Drugs may be classified using a combination of active ingredient and strength. In this case, when, for example, the active ingredient is “metformin” and the strength is 500 mg, drugs are classified as the same drug regardless of whether they are an original drug or a generic drug. Even if the active ingredient of a drug is “metformin”, when the strength is “250 mg”, the drug is classified as a separate drug to “metformin, 500 mg”. Drugs may be classified using a combination of strength and whether they are original or generic. In this case, when, for example, the active ingredient is “metformin”, the strength is 500 mg, and it is an original drug, drugs are classified as the same drug. Even if a drug is “metformin, 500 mg”, when it is a generic drug, it is classified as a separate drug to an original drug.

A disease may be classified by any particle size. For example, a disease may be classified according to the ICD (the International Statistical Classification of Diseases and Related Health Problems)-10 Code (for example, “M600”), and may be classified by integrating a plurality of related ICD-10 Code units (for example, “rheumatoid arthritis”).

Indication data 410 expresses a disease for which a drug has been confirmed to be effective (so-called indication). The medical facility data 400 may be generated based on information provided by, for example, a pharmaceutical company or a government agency. Indication data 410 has an entry per drug.

A column 411 represents an identifier for uniquely identifying a drug. A column 412 represents an indication for each drug. A drug may have only one indication, as for a drug A, or a drug may have a plurality of indications, as for a drug B. In the following description, a drug having only one indication is called an exclusive drug, and a drug having a plurality of indications is called a general drug. The distinction between an exclusive drug and a general drug can change according to a particle size of a disease. A particle size of an indication illustrated in the column 412 has a same particle size as a disease illustrated in the column 403 in the medical facility data 400.

Drug usage amount data 420 represents a usage amount of each of a plurality of drugs in a medical facility. The drug usage amount data 420 may be generated by, for example, an interview survey of a medical facility, or analysis of dispensing reports. The drug usage amount data 420 has an entry per medical facility. Descriptions of a column 421 and a column 422 are omitted because they are similar to the column 401 and the column 401. A number of patients of each disease in a medical facility included in the drug usage amount data 420 is considered to be unclear. Therefore, for these medical facilities, a number of patients of each disease is estimated based on a usage amount of a plurality of drugs.

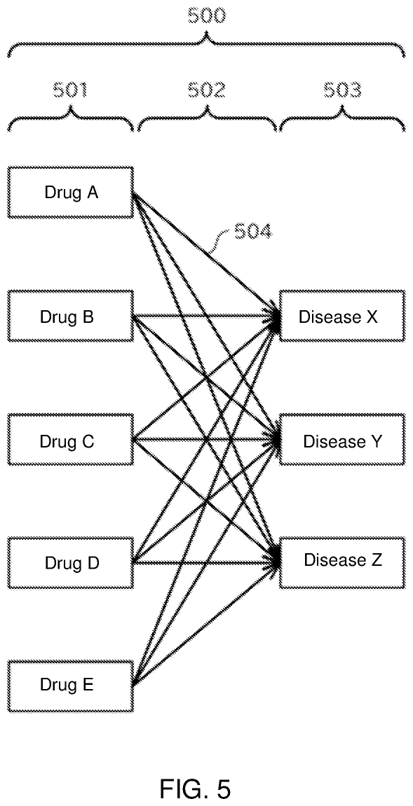

Next, one example of a model 500 created by the learning phase is described with reference to FIG. 5. In one embodiment, the generated model 500 is a linear regression model. In place thereof, another model such as a neural network may be used. A feature value 501 in the model 500 is a usage amount of a drug. The feature value 501 is data input to the model 500. A usage amount of each of a plurality of drugs is used as the feature value 501. An objective variable 503 in the model 500 is a number of patients of a disease. The objective variable 503 in the model 500 is data output from the model 500. A number of patients of each of a plurality of diseases is used as the objective variable 503. A parameter 502 in the model 500 is a coefficient that defines a relationship between the feature value 501 and the objective variable 503. The parameter 502 is also called a weight. In the model 500, the parameter 502 is assigned individually from each of a plurality of the feature value 501 to each of a plurality of the objective variables 503. The machine learning unit 202 determines the parameter 502 in the model 500 by performing machine learning using the medical facility data 400 as the training data.

The machine learning unit 202 generates the model 500 for each disease group. Therefore, the model 500 can be said to be a model unique to a disease group. For example, one defined disease group may be constituted of three diseases, a disease X to a disease Z. In this case, a model (the model 500 in FIG. 5) in which a number of patients of each of the diseases X to Z is made to be the objective variable 503, and a usage amount of each of drugs A to E relating to at least one of the diseases X to Z is made to be the feature value 501 is a model unique to this defined disease group. A drug relating to a disease may be a drug including in an indication the disease as represented by, for example, the indication data 410 in FIG. 4.

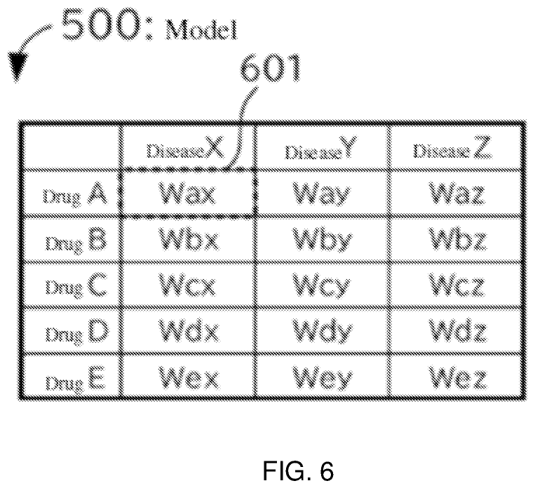

FIG. 6 is a diagram representing the model 500 in a tabular form. The cells in FIG. 6 correspond one-to-one to an arrow in FIG. 5. For example, a cell 601 illustrating a parameter between a usage amount of the drug A and a number of patients of the disease X corresponds to an arrow 504 in FIG. 5.

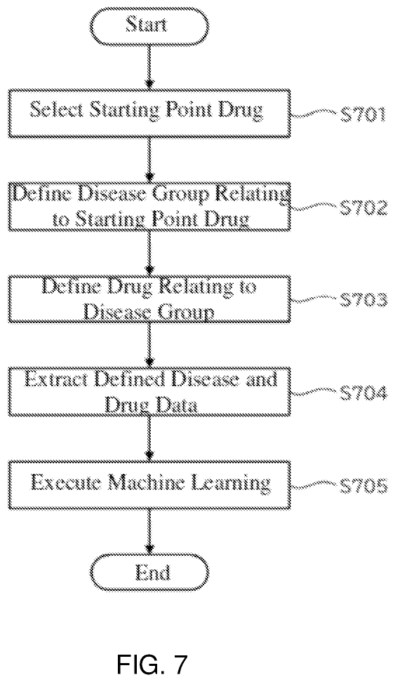

Next, an operational example of the information processing device 100 executing the learning phase is described with reference to FIG. 7. When initiating this operation, the information processing device 100 is enabled to use the medical facility data 400 and the indication data 410. The operation in FIG. 7 may be initiated according to an instruction from a user of the information processing device 100.

In a step S701, the training data generating unit 201 selects a drug to become a starting point for defining a disease group (starting point drug hereinbelow). The starting point drug may be selected according to an instruction from a user of the information processing device 100. The starting point drug may have the same classification as the drugs represented in the column 402 of the medical facility data 400, or it may have a higher or lower classification thereof.

In a step S702, the training data generating unit 201 defines a disease group including a plurality of diseases relating to the starting point drug. For example, when “Humira” is selected as the starting point drug, a disease group including a plurality of diseases relating to an autoimmune disease (rheumatoid arthritis, Crohn's disease, or the like) is defined. The plurality of diseases defined here may have a same particle size as a disease represented in the column 403 of the medical facility data 400. The plurality of diseases relating to the starting point drug may be an indication of a starting point drug as represented by the indication data 410. For example, the diseases X to Z are defined from among the diseases represented in the column 403 of the medical facility data 400.

In a step S703, the training data generating unit 201 defines a drug relating to any of the plurality of diseases defined in the step S702. The plurality of drugs defined here may have a same particle size as the drugs represented in the column 402 of the medical facility data 400. The drug relating to the disease may be a drug having an indication represented by the indication data 410. Of the drugs represented in the column 402 of the medical facility data 400, the drugs A to E are defined for the diseases X to Z.

In a step S704, the training data generating unit 201 generates training data by extracting from the medical facility data 400 a number of patients of each of the plurality of diseases included in the disease group defined in the step S702 and a usage amount of each of the plurality of drugs defined in the step S703. In the training data, a usage amount of a drug becomes the feature value, and a number of patients of a disease becomes the objective variable.

In a step S705, the machine learning unit 202 creates the model 500 by performing machine learning using training data generated in the step S704. Specifically, the machine learning unit 202 determines the parameter 502 of the model 500. Because a deterministic algorithm of the parameter 502 may be the same as an existing algorithm, detailed description thereof is omitted.

A model unique to one disease group is generated by executing the above steps S701 to S705. The information processing device 100 may repeatedly execute the above steps S701 to S705 to generate a model unique to a separate disease group. Further, in the method in FIG. 7, a disease group is defined using a starting point drug. In place thereof, a plurality of diseases constituting one disease group may be defined by a user of the information processing device 100.

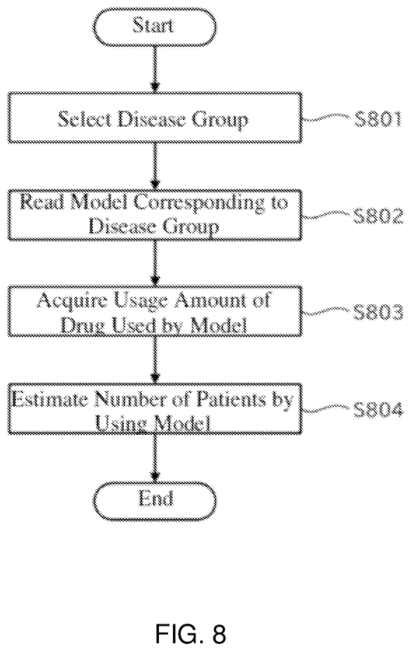

Next, an operational example of the information processing device 100 executing the estimating phase is described with reference to FIG. 8. When initiating this operation, the information processing device 100 is enabled to use the model 500 and the drug usage amount data 420. The operation in FIG. 8 may be initiated according to an instruction from a user of the information processing device 100, and may be initiated periodically (for example, every time the drug usage amount data 420 is updated).

In a step S801, the disease group selecting unit 301 selects a disease group including a disease to be estimated. The disease group may be selected according to an instruction from a user of the information processing device 100, or may be selected according to a prior setting. When a plurality of disease groups is selected, steps S802 to S804 below are executed for each disease group. A disease group selected in this step corresponds to a disease group in a model generated in the learning phase.

In the step S802, the model acquisition unit 302 acquires a model unique to the selected disease group. The model may read from the storage device 106 of the information processing device 100 or may read from a different external storage device than the information processing device 100.

In the step S803, the drug usage amount acquisition unit 303 acquires a usage amount of each of a plurality of drugs used as the feature value of the model. Specifically, the drug usage amount acquisition unit 303 extracts a column to be used as the feature value of the model from among the drug usage amount data 420. A matrix representing this usage amount of a drug per medical facility is made to be M. Each row of M corresponds to a medical facility, and each column of M corresponds to a usage amount of a drug.

In the step S804, the patient number estimating unit 304 estimates a number of patients per medical facility and per disease using the model. A matrix representing the model is made to be W. As illustrated in FIG. 6, each row of W corresponds to a usage amount of a drug, and each column of W corresponds to a number of patients. An estimated value of the number of patients is calculated by M x W. Each row of M x W corresponds to a medical facility, and each column of M x W corresponds to a number of patients.

According to the above embodiment, a number of patients can be accurately estimated according to distribution of a usage amount of a drug in an individual medical facility. Further, machine learning is performed using training data generated by extracting, from the medical facility data 400, a number of patients of a plurality of diseases included in one disease group and a usage amount of a drug relating to this plurality of diseases. Therefore, the accuracy of a model can be improved compared to when machine learning is performed using an entirety of the medical facility data 400 as training data.

First Variation

A variation of the above embodiment is described. The following description focuses on differences from the above embodiment, and matters not described may be similar to the above embodiment.

In the above embodiment, a defined disease group is constituted of the diseases X to Z, and the drugs relating to these diseases are the drugs A to E. Of these drugs, a portion of the drugs (drug B) relates to all of the diseases X to Z, and the other drugs relate only to a portion of the diseases X to Z. In this variation, the machine learning unit 202 performs machine learning using this prior knowledge.

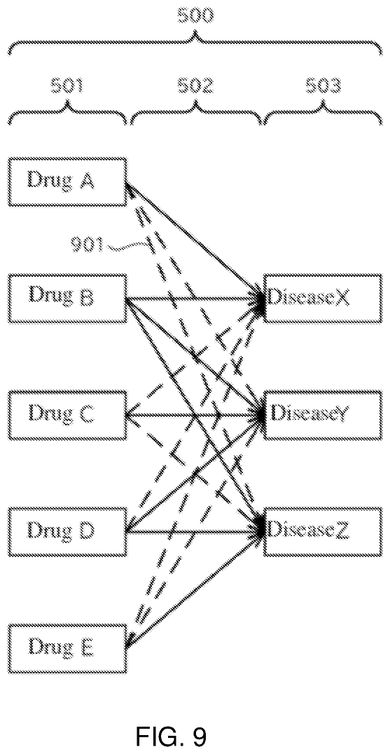

For example, in FIG. 9, of the arrows illustrating the parameters 502 of the model 500, arrows associating an individual drug and a disease, of the plurality of diseases, not related to the individual drug are illustrated as a dotted line. For example, as illustrated in the indication data 410 in FIG. 4, a drug A relates only to the disease X of the diseases X to Z. Therefore, only the arrow drawn from the drug A to the disease X is illustrated as a solid line, and the arrows drawn from the drug A to the disease Y and the disease Z are illustrated as a dotted line. During machine learning, the machine learning unit 202 imposes a penalty on a parameter relating to a pair illustrated by a dotted line. For example, the machine learning unit 202 may always make the values of these parameters zero. In place thereof, the machine learning unit 202 may assign an upper limit to these parameters.

By imposing a penalty in this manner, accuracy of the machine learning can be further improved.

Second Variation

A variation of the above embodiment is described. The following description focuses on differences from the above embodiment. Matters which are not described may be similar to the above embodiment. In the above embodiment, when a disease group is a same disease group in the estimating phase, a same model is used (for example, the model 500) for a plurality of medical facilities targeted for estimation. In this variation, a number of patients is estimated after making individual adjustments to this model for each medical facility. In the following description, as in the model 500, a model used to estimate a number of patients is unique to one defined disease group and is called a global model. In this variation, a special model for adjusting the global model is further generated by machine learning. As illustrated in the indication data 410 in FIG. 4, drugs are classified as exclusive drugs (for example, drugs A, C, E, and G) or general drugs (for example, drugs B and D). In this variation, a special model is generated using this prior knowledge.

In the learning phase, following the step S703 in FIG. 7, the training data generating unit 201 acquires training data including one general drug relating to two or more diseases of a plurality of diseases included in one defined disease group, a usage amount of each of one or more exclusive drugs relating to only one of any of the two or more diseases relating to the one general drug, and a number of patients of each of the two or more diseases. This training data becomes a subset of training data generated in the step S704.

For example, similarly to the above embodiment, a defined disease group is made to be constituted of the diseases X to Z. First, the training data generating unit 201 selects the drug D from among the drugs B and D relating to two or more diseases of the diseases X to Z. Next, the training data generating unit 201 defines the drugs C and E, which relate to only one of either of the diseases Y or Z related by this drug D. The training data generating unit 201 acquires training data from the medical facility data 400 by extracting a column corresponding to the drugs C to E and the diseases Y and Z.

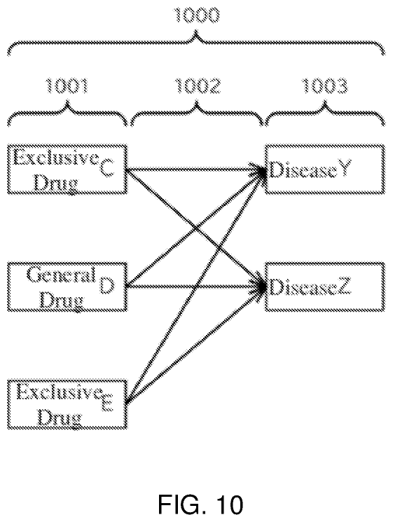

Afterwards, the machine learning unit 202 generates a model 1000 wherein, as illustrated in FIG. 10, by performing machine learning using this training data, a usage amount of each of the drugs C to E is made to be a feature value 1001 and a number of patients of each of the diseases Y and Z is made to be an objective variable 1003. The model 1000 is the special model above. A parameter 1002 of the model 1000 is a coefficient that defines a relationship between the feature value 1001 and the objective variable 1003. The model 1000 is a linear regression model. In place thereof, another model such as a neural network may be used. The model 1000 is generated for one selected general drug (the drug D in the above example). Therefore, the model 1000 can be said to be a model unique to one general drug D. The machine learning unit 202 can generate a special model that is also unique to a separate general drug B.

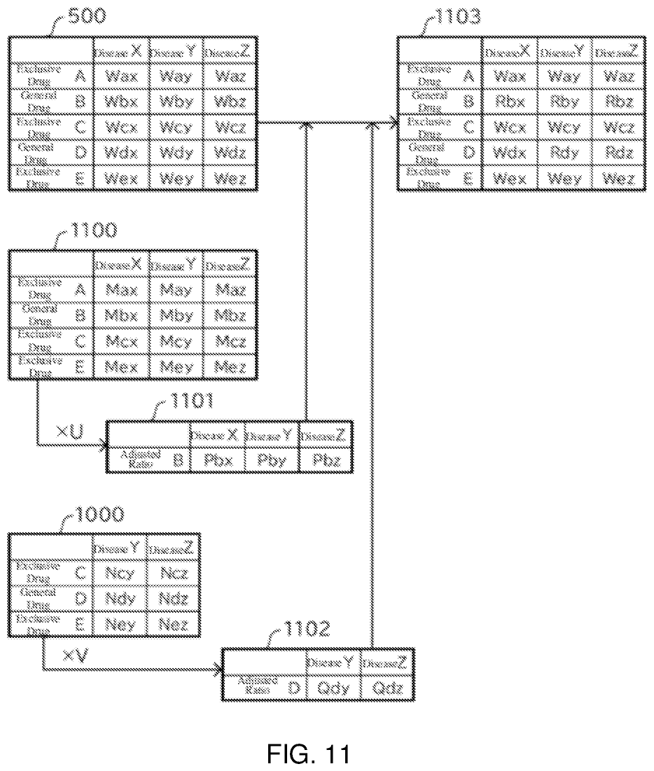

In the estimating phase, during the step S803 and the step S804 in FIG. 8, a global model read in the step S802 is adjusted using the special model. An adjustment method is described in detail with reference to FIG. 11. As described above, the model 500 in FIG. 11 is a tabular representation of a global model unique to a disease group constituted of the diseases X to Z. A matrix corresponding to the model 500 is made to be W. Further, the model 1100 is a tabular representation of a special model unique to the general drug B. A matrix corresponding to the model 1100 is made to be M. The model 1000 is a tabular representation of a special model unique to the general drug D. A matrix corresponding to the model 1000 is made to be N.

The patient number estimating unit 304 selects one medical facility (one entry of the drug usage amount data 420) to estimate a number of patients and acquires a usage amount of each of a plurality of the drugs A to C and E used as the feature value of the model 1100 for the selected defined medical facility. Specifically, the patient number estimating unit 304 extracts a column used as the feature value of the model 1100 from among the drug usage amount data 420. A row vector representing a usage amount of a drug for this defined medical facility is made to be U. The patient number estimating unit 304 estimates a number of patients of the diseases X to Z relating to the general drug B in this defined medical facility using the model 1100. This number of patients is calculated by U×M. A column vector 1101 representing this estimated number is made to be P (that is, P=U×M). This column vector 1101 is considered to represent a ratio (Pbx:Pby:Pbz) of the number of patients of the plurality of diseases X to Z for which the general drug B is used in one defined medical facility.

Then, the patient number estimating unit 304 adjusts a parameter of the row for the general drug B in the model 500 such that, in the model 500, a ratio of the parameter of the row for the general drug B matches the ratio of the number of patients in the column vector 1101. For example, the patient number estimating unit 304 replaces a coefficient Wbx between the general drug B and the disease X with Rbx=(Wbx+Wby+Wbz) x Pbx/(Pbx+Pby+Pbz). Similarly, the patient number estimating unit 304 replaces Wby and Wbz in the model 500 with Rby and Rbz.

Further, the patient number estimating unit 304 acquires a usage amount of each of a plurality of the drugs C to E used as the feature value of the model 1000 for the selected defined medical facility. A row vector representing a usage amount of a drug for this defined medical facility is made to be V. The patient number estimating unit 304 estimates a number of patients of the diseases Y and Z relating to the general drug D in this defined medical facility using the model 1000. This number of patients is calculated by V x N. A column vector 1102 representing this estimated number is made to be Q. This column vector 1102 is considered to represent a ratio (Qdy:Qdz) of a number of patients of a plurality of the diseases Y and Z for which the general drug D is used in one defined medical facility.

Then, the patient number estimating unit 304 adjusts a parameter of the row for the general drug D in the model 500 such that, in the model 500, a ratio of the parameter of the row for the general drug D matches the ratio of the number of patients in the column vector 1102. For example, the patient number estimating unit 304 replaces a coefficient Wdy between the general drug D and the disease Y with Rdy=(Wdy+Wdz) x Qdy/(Qdy+Qdz). Similarly, the patient number estimating unit 304 replaces Wdz in the model 500 with Rdz.

A model obtained by performing an adjustment such as the above is made to be a model 1103. Because column vectors U and V differ for each medical facility, the model 1103 also differs for each medical facility. In the above step S804, the patient number estimating unit 304 performs estimation of the number of patient using the model 1103 in place of the model 500.

As such, estimating accuracy of a number of patients can be improved by using a model unique to a general drug.

The invention is not limited to the above embodiments, and a variety of variations and changes are possible within the scope of the gist of the invention.

What is claimed is:

1. A non-transitory computer readable medium encoded with a computer program that comprises instructions to cause a processor to:
train a global model to correlate between amounts of medicines administered to patients of multiple medical facilities for each of multiple medical conditions, and numbers of patients treated for each of the medical conditions by the respective medical facilities; and
use the global model to estimate numbers of patients treated for each of the medical conditions at the selected medical facility based on amounts of the medicines administered by the selected medical facility.

2. The non-transitory computer readable medium of claim 1, further comprising instructions to cause the processor to:
tailor training of the global model for a situation in which a first one of the medicines is administered for a plurality of the medical conditions and a second one of the medicines is administered for a subset of the plurality of medical conditions.

3. The non-transitory computer readable medium of claim 2, further comprising instructions to cause the processor to:
impose a penalty on parameters of the global model that relate a medicine of the subset to patients for whom the medicine of the subset is not administered.

4. The non-transitory computer readable medium of claim 1, further comprising instructions to cause the processor to:
tailor the global model for a selected one of the medical facilities.

5. The non-transitory computer readable medium of claim 4, further comprising instructions to cause the processor to:
modify parameters of the global model for the selected medical facility based on a ratio at which one or more of the medicines are administered by the selected medical facility.

6. The non-transitory computer readable medium of claim 5, wherein the medicines include a general medicine administered for a plurality of the medical conditions, and wherein the medicines further include one or more exclusive medicines, each administered for a respective one of the plurality of medical conditions, further comprising instructions to cause the processor to:
train an adjustment model to determine a ratio at which the selected medical facility administers the general medicine amongst patients of the plurality of diseases; and
modify the parameters of the global model based on the determined ratio.

7. The non-transitory computer readable medium of claim 6, further comprising instructions to cause the processor to:
train the adjustment model to correlate between amounts of the general medicine and amounts of the one or more exclusive medicines administered by the multiple medical facilities, and numbers of patients treated for each medical condition of the subset of medical conditions by the multiple medical facilities;
provide the adjustment model with amounts of the general medicine and amounts of the one or more exclusive medicines administered by the selected medical facility to estimate a number of patients treated for each medical condition of the subset of medical conditions by the selected medical facility; and
determine the ratio based on the estimated number of patients treated for each medical condition of the subset of medical conditions by the selected medical facility.

8. An apparatus, comprising a processor and memory configured to:
train a global model to correlate between amounts of medicines administered to patients of multiple medical facilities for each of multiple medical conditions, and numbers of patients treated for each of the medical conditions by the respective medical facilities; and
use the global model to estimate numbers of patients treated for each of the medical conditions at the selected medical facility based on amounts of the medicines administered by the selected medical facility.

9. The apparatus of claim 8, wherein the processor and memory are further configured to:
tailor training of the global model for a situation in which a first one of the medicines is administered for a plurality of the medical conditions and a second one of the medicines is administered for a subset of the plurality of medical conditions.

10. The apparatus of claim 9, wherein the processor and memory are further configured to:
impose a penalty on parameters of the global model that relate a medicine of the subset to patients for whom the medicine of the subset is not administered.

11. The apparatus of claim 8, wherein the processor and memory are further configured to:
tailor the global model for a selected one of the medical facilities.

12. The apparatus of claim 11, wherein the processor and memory are further configured to:
modify parameters of the global model for the selected medical facility based on a ratio at which one or more of the medicines are administered by the selected medical facility.

13. The apparatus of claim 13, wherein the medicines include a general medicine administered for a plurality of the medical conditions, and wherein the medicines further include one or more exclusive medicines, each administered for a respective one of the plurality of medical conditions, wherein the processor and memory are further configured to:
train an adjustment model to determine a ratio at which the selected medical facility administers the general medicine amongst patients of the plurality of diseases; and
modify the parameters of the global model based on the determined ratio.

14. The apparatus of claim 13, wherein the processor and memory are further configured to:
train the adjustment model to correlate between amounts of the general medicine and amounts of the one or more exclusive medicines administered by the multiple medical facilities, and numbers of patients treated for each medical condition of the subset of medical conditions by the multiple medical facilities;
provide the adjustment model with amounts of the general medicine and amounts of the one or more exclusive medicines administered by the selected medical facility to estimate a number of patients treated for each medical condition of the subset of medical conditions by the selected medical facility; and
determine the ratio based on the estimated number of patients treated for each medical condition of the subset of medical conditions by the selected medical facility.

15. A method, comprising:
training a global model to correlate between amounts of medicines administered to patients of multiple medical facilities for each of multiple medical conditions, and numbers of patients treated for each of the medical conditions by the respective medical facilities; and
using the global model to estimate numbers of patients treated for each of the medical conditions at the selected medical facility based on amounts of the medicines administered by the selected medical facility.

16. The method of claim 15, further comprising:
tailoring training of the global model for a situation in which a first one of the medicines is administered for a plurality of the medical conditions and a second one of the medicines is administered for a subset of the plurality of medical conditions.

17. The method of claim 16, wherein the tailoring comprises:
imposing a penalty on parameters of the global model that relate a medicine of the subset to patients for whom the medicine of the subset is not administered.

18. The method of claim 15, further comprising:
tailoring the global model for a selected one of the medical facilities.

19. The method of claim 18, wherein the tailoring comprises:
modifying parameters of the global model for the selected medical facility based on a ratio at which one or more of the medicines are administered by the selected medical facility.

20. The method of claim 19, wherein the medicines include a general medicine administered for a plurality of the medical conditions, and wherein the medicines further include one or more exclusive medicines, each administered for a respective one of the plurality of medical conditions, wherein the tailoring further comprises:
training an adjustment model to determine a ratio at which the selected medical facility administers the general medicine amongst patients of the plurality of diseases; and
performing the modifying the parameters of the global model based on the determined ratio.
